SEQUENCE LISTING (25 sequences)

SEQ ID NO: 1 - DNA, 24 bp, Artificial Sequence
  Description of Artificial Sequence Synthetic primer
caatgttgttccttgaggaagttg

SEQ ID NO: 2 - DNA, 20 bp, Artificial Sequence
  Description of Artificial Sequence Synthetic primer
gttgttccttgaggaagttg

SEQ ID NO: 3 - DNA, 17 bp, Artificial Sequence
  Description of Artificial Sequence Synthetic primer
gttccttgaggaagttg

SEQ ID NO: 4 - DNA, 41 bp, Artificial Sequence
  Description of Artificial Sequence Synthetic primer
cgattccgctccagacttctcgggaacaaagaaggcatcgt

SEQ ID NO: 5 - DNA, 41 bp, Artificial Sequence
  Description of Artificial Sequence Synthetic primer
accgcatcgaatgcatgtctcgggtggcagcattgttatta

SEQ ID NO: 6 - DNA, 40 bp, Artificial Sequence
  Description of Artificial Sequence Synthetic primer
acgttagccaccatacggatacccaaagaccacattggca

SEQ ID NO: 7 - DNA, 40 bp, Artificial Sequence
  Description of Artificial Sequence Synthetic primer
actgatccgcactaacgactacccaaagaccacacggact

SEQ ID NO: 8 - DNA, 40 bp, Artificial Sequence
  Description of Artificial Sequence Synthetic primer
acgttagccaccatacttgaacccaaagaccacattggca

SEQ ID NO: 9 - DNA, 126 bp, Human Severe Acute Respitory Syndrome Coronavirus
aacaaagaaggcatcgtatgggttgcaactgagggagccttgaatacacccaaagaccacattggcacccgcaatcctaataacaatgctgccaccgtgctacaacttcctcaaggaacaacattg

SEQ ID NO: 10 - RNA, 126 nt, Human Severe Acute Respitory Syndrome Coronavirus
aacaaagaaggcaucguauggguugcaacugagggagccuugaauacacccaaagaccacauuggcacccgcaauccuaauuacaaugcugccaccgugcuacaacuuccucaaggaacaacauug

SEQ ID NO: 11 - DNA, 126 bp, Human Severe Acute Respitory Syndrome Coronavirus
aacaaagaaggcatcgtatgggttgcaactgagggagccttgaatacacccaaagaccacacggactcccgcaatcctaattacaatgctgccaccgtgctacaacttcctcaaggaacaacattg

SEQ ID NO: 12 - RNA, 126 nt, Human Severe Acute Respitory Syndrome Coronavirus
aacaaagaaggcaucguauggguugcaacugagggagccuugaauacacccaaagaccacacggacucccgcaauccuaauuacaaugcugccaccgugcuacaacuuccucaaggaacaacauug

SEQ ID NO: 13 - DNA, 28 bp, Artificial Sequence
  Description of Artificial Sequence Synthetic probe
tcccgagtacgttagccaccatacggat

SEQ ID NO: 14 - DNA, 28 bp, Artificial Sequence
  Description of Artificial Sequence Synthetic probe
acccgagtagctatccgccataagccat

SEQ ID NO: 15 - DNA, 29 bp, Artificial Sequence
  Description of Artificial Sequence Synthetic probe
tccccgagtacgttagccaccatacttga

SEQ ID NO: 16 - DNA, 29 bp, Artificial Sequence
  Description of Artificial Sequence Synthetic probe
tccccgagtactgatccgcactaacgact

SEQ ID NO: 17 - DNA, 22 bp, Artificial Sequence
  Description of Artificial Sequence Synthetic primer
atgttcccgaaggtgtgacttc

SEQ ID NO: 18 - DNA, 41 bp, Artificial Sequence
  Description of Artificial Sequence Synthetic primer
cgattccgctccagacttctcgggacaaggaactgattaca

SEQ ID NO: 19 - DNA, 41 bp, Artificial Sequence
  Description of Artificial Sequence Synthetic primer
accgcatcgaatgcatgtctcgggtgcgtgacattccaaag

SEQ ID NO: 20 - DNA, 40 bp, Artificial Sequence
  Description of Artificial Sequence Synthetic primer
acgttagccaccatacggatcaatttgctccaagtgcctc

SEQ ID NO: 21 - DNA, 40 bp, Artificial Sequence
  Description of Artificial Sequence Synthetic primer
acgttagccaccatacttgacaatttgctccaagtgcctc

SEQ ID NO: 22 - DNA, 111 bp, Human Severe Acute Respitory Syndrome Coronavirus
gacaaggaactgattacaaacattggccgcaaattgcacaatttgctccaagtgcctctgcattctttggaatgtcacgcattggcatggaagtcacaccttcgggaacat

SEQ ID NO: 23 - RNA, 111 nt, Human Severe Acute Respitory Syndrome Coronavirus
gacaaggaacugauuacaaacauuggccgcaaauugcacaauuugcuccaagugccucugcauucuuuggaaugucacgcauuggcauggaagucacaccuucgggaacau

SEQ ID NO: 24 - DNA, 15 bp, Artificial Sequence
  Description of Artificial Sequence Synthetic primer
gcctcttcgctatta

SEQ ID NO: 25 - DNA, 40 bp, Artificial Sequence
  Description of Artificial Sequence Synthetic primer
agctatccgccataagccatacccaaagaccacacggact

(19) United States
(12) Patent Application Publication
(10) Pub. No.: US 2010/0136513 A1
(43) Pub. Date: Jun. 3, 2010

(54) Assay for sars coronavirus by amplification and detection of nucleocapsid rna sequence

(76) Inventors: Jianrong Lou, Boyds, MD (US); James A. Price, JR., Lutherville, MD (US); Daretta A. Yursis, Parkton, MD (US); David M. Wolfe, Red Lion, PA (US); Lisa M. Keller, York, PA (US); Tobin J. Hellyer, Westminster, MD (US)

Correspondence Address: David W. Highet;Becton, Dickson and company, 1 Becton Drive, Mail Code 110, Franklin Lakes, NJ, 07417-1880, US

(21) Appl. No.: 10/570,781

(22) PCT Filed: Sep. 13, 2004
(86) PCT No.: PCT/US04/29692

§ 371 Date: Mar. 21, 2007

Related U.S. Application Data
(60) Provisional application No. 60/502,278, filed on Sep. 12, 2003.

(51) Int. Cl. C12Q 1/70; C07H 21/04; C12P 19/34; C12Q 1/68

(52) U.S. Cl. 435/5; 536/24.33; 435/91.2; 435/6

(57) ABSTRACT

Primers and probes derived from SARS-CoV nucleic acid that facilitate detection and/or quantification of the nucleocapsid gene are disclosed. The disclosed sequences may be used in a variety of amplification and non-amplification formats for detection of SARSCoV infection.

The present application claims priority to U.S. Provisional Application Ser. No. 60/502,278, filed Sep. 12, 2003, which is herein incorporated by reference in its entirety.

### TECHNICAL FIELD

The present invention relates to methods to assay for the presence of Severe Acute Respiratory Syndrome coronavirus by amplification and detection of the nucleocapsid RNA sequence.

### BACKGROUND ART

Severe acute respiratory syndrome (SARS) is a recently emerging disease associated with atypical pneumonia in infected patients. The disease is unusually severe, and there is no known treatment. The incubation period for SARS is typically between 2 and 10 days. Sympathkumar et al., Mayo Clin. Proc. 78: 882-890 (2003). Physical manifestations of SARS include fever, followed by a dry, nonproductive cough and shortness of breath. Death from respiratory failure occurs in about 3% to 10% of SARS cases. Centers for Disease Control and Prevention (CDC). Morb. Mortal. Wkly. Report. 52: 357 (2003).

Clinical diagnosis of SARS is often a slow process because initial diagnostic testing of suspected SARS patients includes a chest radiograph, pulse oximetry, blood culture, sputum Gram's stain and culture, and testing for other viral respiratory infections. CDC, Guidelines and Recommendations: Interim Guidelines for Laboratory Diagnosis of SARS-CoV Infection, July (2003). This difficulty is also reflected by the fact that two of the most common diagnostic procedures—detection of serum antibodies to the SARS virus and isolation in cell culture of the virus from a clinical specimen—often take days or even weeks to complete. CDC, Guidelines and Recommendations: Interim Guidelines for Laboratory Diagnosis of SARS-CoV Infection, July (2003). Thus, the need for the establishment of a rapid and noninvasive test for SARS is essential for monitoring and control of the disease.

Early in 2003, a novel coronavirus was identified as the causative agent of SARS. Drosten et al., N. Engl. J. Med. 348: 1967-76 (2003). The coronaviruses are a diverse group of RNA viruses that cause respiratory and enteric diseases in humans and other animals. They are the largest of the RNA viruses, with a genome of approximately 30,000 nucleotides. Rota et al., Science 300:1394-1399 (2003). The SARS-Coronavirus (SARS-CoV) is an enveloped, positive-stranded RNA virus. Based on sequence analysis, SARS-CoV is a member of a new group of coronavirus (order Nidovirales, Family Coronaviridae, genus Coronavirus). Rota et al., supra.

The nucleocapsid (N) gene is located towards the 3′ end of the SARS-CoV genome. The subgenomic RNA encoding the N protein is abundantly expressed after viral infection and offers a good target gene for detection of the virus. Rota et al., supra. An assay that tests for the presence of the viral nucleic acid would allow for the rapid and sensitive detection of SARS-CoV. Such an assay would provide a more sensitive alternative to serological testing, direct fluorescent antibody staining or urinary antigen testing.

### DISCLOSURE OF THE INVENTION

According to one aspect, the present invention provides an oligonucleotide set comprising a first amplification primer and a second amplification primer, the first amplification primer selected from the group consisting of SEQ ID NOs.: 4 and 18 and the second amplification primer selected from the group consisting of SEQ ID NOs.: 5 and 19. In another aspect, the first amplification primer consists essentially of SEQ ID NO.: 4 and the second amplification primer consists essentially of SEQ ID NO.: 5. In yet another aspect of the present invention, the first amplification primer consists essentially of SEQ ID NO.: 18 and the second amplification primer consists essentially of SEQ ID NO.: 19.

According to an additional aspect, the present invention provides an oligonucleotide set comprising a first amplification primer and a second amplification primer, the first amplification primer selected from the group consisting of the target binding sequence of SEQ ID NOs.: 4 and 18 and the second amplification primer selected from the group consisting of the target binding sequences SEQ ID NOs.: 5 and 19. In another aspect, the first amplification primer consists essentially of the target binding sequence of SEQ ID NO.: 4 and the second amplification primer consists essentially of the target binding sequence of SEQ ID NO.: 5. In yet another aspect of the present invention, the first amplification primer consists essentially of the target binding sequence of SEQ ID NO.: 18 and the second amplification primer consists essentially of the target binding sequence of SEQ ID NO.: 19.

According to a further aspect, the oligonucleotide set further comprises a signal primer and a reporter probe, the signal primer selected from the group consisting of the target binding sequences of SEQ ID NOs.: 6, 8, 20 and 21 and the reporter probe selected from the group consisting of SEQ ID NOs.: 13 and 15. In one aspect, the signal primer consists essentially of the target binding sequence of SEQ ID NO.: 6 and the reporter probe consists essentially of SEQ ID NO.: 13. In yet another aspect, the signal primer consists essentially of the target binding sequence of SEQ ID NO.: 8 and the reporter probe consists essentially of SEQ ID NO.: 15. According to a further aspect, the oligonucleotide set further comprises a second signal primer and a second reporter probe, the second signal primer consisting essentially of SEQ ID NO.: 25 and the second reporter probe consisting essentially of SEQ ID NO.: 14. In a further embodiment, the oligonucleotide set with a second signal primer and a second reporter probe further comprises one or more bumper primers selected from the group consisting of SEQ ID NOs.: 1, 2, 3 and 17.

In an additional embodiment, the signal primer consists essentially of the target binding sequence of SEQ ID NO.: 20 and the reporter probe consists essentially of SEQ ID NO.: 13. In yet another embodiment of the present invention, the signal primer consists essentially of the target binding sequence of SEQ ID NO.: 21 and the reporter probe consists essentially of SEQ ID NO.: 15. In a further aspect, the oligonucleotide set further comprises a second signal primer and a second reporter probe, the second signal primer consisting essentially of SEQ ID NO.: 7 and the second reporter probe consisting essentially of the hybridization sequence of SEQ ID NO.: 16. In still another aspect, the oligonucleotide set comprising a second signal primer and a second reporter probe further comprises one or more bumper primers selected from the group consisting of SEQ ID NOs.: 1, 2, 3 and 17.

According to a further aspect, the target binding sequences of SEQ ID NOs.: 4, 5, 18 and 19 comprise a sequence required for an amplification reaction. In another embodiment of the present invention, the sequence required for the amplification reaction comprises a restriction endonuclease recognition site that is nickable by a restriction endonuclease. In yet another embodiment, the sequence required for the amplification reaction comprises a promoter recognized by an RNA polymerase. In still another embodiment, the hybridization sequences of SEQ ID NOs.: 6, 8, 13, 15, 20 and 21 further comprise an indirectly detectable marker. In another aspect, the indirectly detectable marker comprises an adapter sequence.

In a further embodiment, the present invention provides an oligonucleotide comprising a SARS-CoV target sequence selected from the group consisting of SEQ ID NOs.: 9, 10, 22 and 23.

In another embodiment, the present invention provides a method for detecting the presence or absence SARS-CoV in a sample, the method comprising: (a) treating the sample with a plurality of nucleic acid primers in a nucleic acid amplification reaction wherein a first primer is selected from the group consisting of the target binding sequences of SEQ ID NO.: 4 and SEQ ID NO.: 18 and a second primer is selected from the group consisting of the target binding sequences of SEQ ID NO.: 5 and SEQ ID NO.: 19; and (b) detecting any amplified nucleic acid product, wherein detection of the amplified product indicates presence of SARS CoV. In a further embodiment, the first primer consists essentially of SEQ ID NO.: 4 and the second primer consists essentially of SEQ ID NO.: 5. In yet another embodiment, the first primer consists essentially of SEQ ID NO.: 18 and the second primer consists essentially of SEQ ID NO.: 19. In still another embodiment, step (a) comprises a Strand Displacement Amplification (SDA) reaction. In a further embodiment, the SDA reaction utilizes one or more bumper primers selected from the group consisting of SEQ ID NOs.: 1, 2, 3 and 17. In yet another embodiment, the SDA reaction comprises a thermophilic Strand Displacement Amplification (tSDA) reaction. In an additional embodiment, the tSDA reaction is a homogeneous fluorescent real time tSDA reaction. In a further embodiment, step (b) includes the step of hybridizing said amplified nucleic acid product with a signal primer selected from the group consisting of SEQ ID NOs.: 6, 8, 20 and 21.

According to a further aspect, the present invention provides a method for amplifying a target nucleic acid sequence of SARS-CoV comprising: (a) hybridizing to the nucleic acid (i) a first amplification primer selected from the group consisting of the target binding sequences of SEQ ID NO.: 4 and 18; and (ii) a second amplification primer selected from the group consisting of the target binding sequences of SEQ ID NO.: 5 and 19; and (b) extending the hybridized first and second amplification primers on the target nucleic acid sequence whereby the target nucleic acid sequence is amplified. According to a further aspect of the method, the first amplification primer consists essentially of the target binding sequence of SEQ ID NO.: 4 and the second amplification primer consists essentially of the target binding sequence of SEQ ID NO.: 5. According to a further aspect of the method, the first amplification primer consists essentially of the target binding sequence of SEQ ID NO.: 18 and the second amplification primer consists essentially of the target binding sequence of SEQ ID NO.: 19.

In still another aspect of the method, the target binding sequences of SEQ ID NO.: 4 and SEQ ID NO.: 5 comprise a sequence required for an amplification reaction. In another embodiment, the sequence required for the amplification reaction comprises a restriction endonuclease recognition site that is nickable by a restriction endonuclease. In yet another embodiment, the sequence required for the amplification reaction comprises a promoter recognized by an RNA polymerase.

In yet another aspect of the invention, the target binding sequences of SEQ ID NO.: 18 and SEQ ID NO.: 19 comprise a sequence required for an amplification reaction. In a further aspect, the sequence required for the amplification reaction comprises a restriction endonuclease recognition site that is nickable by a restriction endonuclease. In a further embodiment, the sequence required for the amplification reaction comprises a promoter recognized by an RNA polymerase.

In a further aspect, the method further comprises indirectly detecting the amplified target nucleic acid by hybridization to a signal primer. In yet another aspect, the signal primer is selected from the group consisting of SEQ ID NOs.: 6, 8, 20 and 21.

According to a further aspect, the target nucleic acid sequence is selected from the group consisting of SEQ ID NOs.: 9, 10, 22 and 23.

According to a further aspect, the present invention provides a method of quantifying the amount of SARS-CoV nucleic acid in a target sample comprising the steps of: a) combining the target sample with a known concentration of SARS-CoV internal control nucleic acid; b) amplifying the target nucleic acid and internal control nucleic acid in an amplification reaction; c) detecting the amplified nucleic acid; and d) analyzing the relative amounts of amplified SARS-CoV target nucleic acid and internal control nucleic acid. In another embodiment, step (b) comprises a strand displacement amplification reaction. In yet another embodiment, the SDA reaction comprises a tSDA reaction. In a further aspect, the amplification reaction utilizes one or more signal primers selected from the group consisting of the hybridization sequences of SEQ ID NOs.: 6, 7, 8, 20, 21, and 25 and one or more reporter probes selected from the group consisting of the hybridization sequences of SEQ ID NOs.: 13, 14, 15 and 16. In yet another aspect, the hybridization sequences of SEQ ID NOs.: 6, 7, 8, 13, 14, 15, 16, 20, 21, and 25 comprise an indirectly detectable marker. In another embodiment, the indirectly detectable marker comprises an adapter sequence.

### MODES FOR CARRYING OUT THE INVENTION

The methods of the present invention are useful for assaying for the presence of SARS-CoV by the amplification and detection of the SARS-CoV nucleocapsid (N) sequence. The primers and probes of the present invention are based on portions of the SARS-CoV nucleocapsid gene. The present invention also provides oligonucleotides that may be used in amplification, detection and/or quantification of the N gene. The oligonucleotides may be used in all types of amplification reactions such as, for example, Strand Displacement Amplification (SDA), Polymerase Chain Reaction (PCR), Ligase Chain Reaction, Nucleic Acid Sequence Based Amplification (NASBA), Rolling Circle Amplification (RCA), Transcription Mediated Amplification (TMA) and QB Replicase-mediated amplification. The present invention further provides oligonucleotides that may be used in amplification, detection and/or quantification of the N gene with sufficient specificity and sensitivity.

The methods of the present invention may be employed, for example, but not by way of limitation, to test clinical specimens obtained from suspected SARS patients. The specimens, or test samples, may be collected from any source suspected of containing SARS nucleic acid. For animals, preferably, mammals, and more preferably, humans, the source of the test samples may include blood, bone marrow, lymph, hard tissues (e.g., liver, spleen, kidney, lung, ovary, etc.), sputum, feces, urine, upper and lower respiratory specimens and other clinical samples. Other sources may include veterinary and environmental samples, as well as in vitro cultures. Those skilled in the art are capable of determining appropriate clinical sources for use in diagnosis of SARS-CoV infection.

### Definitions

The following definitions are provided for reason of clarity, and should not be considered as limiting. Except where noted, the technical and scientific terms used herein are intended to have the same meaning as commonly understood by one of ordinary skill in the art to which the invention pertains.

An “amplification primer” is an oligonucleotide for amplification of a target sequence by extension of the oligonucleotide after hybridization to a target sequence or by ligation of multiple oligonucleotides that are adjacent when hybridized to the target sequence. At least a portion of the amplification primer hybridizes to the target. This portion is referred to as the target binding sequence and it determines target-specificity of the primer. In addition to the target binding sequence, certain amplification methods require specialized non-target binding sequences in the amplification primer. These specialized sequences are necessary for the amplification reaction to proceed and typically serve to append that specialized sequence to the target. For example, but not by of limitation, the amplification primers used in SDA include a restriction endonuclease recognition 5′ to the target binding sequence, as disclosed in U.S. Pat. Nos. 5,455,166 and 5,270,184, each of which is incorporated herein by reference. NASBA, Self-Sustaining Sequence Replication (3SR) and transcription-based amplification primers require an RNA polymerase promoter linked to the target binding sequence of the primer. Linking such specialized sequences to a target binding sequence for use in a selected amplification reaction is routine in the art. In contrast, amplification methods such as PCR, which do not require specialized sequences at the ends of the target, generally employ amplification primers consisting of only target binding sequence.

As used herein, the terms “primer” and “probe” refer to the function of the oligonucleotide. A primer is typically extended by polymerase or ligation following hybridization to the target whereas a probe may either function by hybridization to the target or through hybridization followed by polymerase-based extension. A hybridized oligonucleotide may function as a probe if it is used to capture or detect a target sequence, and the oligonucleotide may function as a primer when it is employed as a target binding sequence in an amplification primer. It will therefore be appreciated that any of the target binding sequences disclosed herein for amplification, detection or quantification of SARS-CoV may be used either as hybridization probes or as target binding sequences in primers for detection or amplification, optionally linked to a specialized

A “bumper” or “external primer” is a primer that anneals to a target sequence upstream of (i.e., 5′ to) an amplification primer, such that extension of the external primer displaces the downstream primer and its extension product, i.e., a copy of the target sequence comprising the SDA restriction endonuclease recognition site is displaced. The bumper primers, therefore, consist only of target binding sequences and are designed so that they anneal upstream of the amplification primers and displace them when extended. External primers are designated B₁ and B₂ by G. Walker, et al., Nuc. Acids Res. 20:1692-1696. Extension of external primers is one method for displacing the extension products of amplification primers, but heating may also be suitable in certain cases.

A “reverse transcription primer” also consists only of target binding sequences. It is hybridized at the 3′ end of an RNA target sequence to prime reverse transcription of the target. Extension of the reverse transcription primer produces a heteroduplex comprising the RNA target and the cDNA copy of the RNA target produced by reverse transcription. The cDNA is separated from the RNA and (e.g., by heating, RNase H, or strand displacement) to make it single-stranded and available for amplification. Optionally, a second reverse transcription primer may be hybridized at the 3′ end of the target sequence in the cDNA to prime second strand synthesis prior to amplification. Optionally, a reverse transcription primer may also function as an amplification or bumper primer.

The terms “target” and “target sequence” refer to nucleic acid sequences (DNA and/or RNA) to be amplified, replicated or detected. These include the original nucleic acid sequence to be amplified and its complementary second strand, as well as either strand of a copy of the original target sequence produced by amplification or replication of the target sequence. “Amplification products,” “extension products” or “amplicons” are oligonucleotides or polynucleotides that comprise copies of the target sequence produced during amplification or replication of the target sequence.

The term “polymerase” refers to any of various enzymes, such as DNA polymerase, RNA polymerase, or reverse transcriptase that catalyze the synthesis of nucleic acids on preexisting nucleic acid templates. A DNA polymerase assembles the DNA from deoxyribonucleotides, while RNA polymerase assembles the RNA from ribonucleotides.

Based on alignment of 25 SARS-CoV nucleotide sequences, two regions were selected as target sequences for use in amplification of the nucleocapsid region, as shown in Tables 1 and 3.

In one embodiment of the present invention, Reverse Transcriptase-Strand Displacement Amplification (RT-SDA) has been adapted for the detection of SARS-CoV nucleocapsid RNA in both genomic and subgenomic RNA sequences. SDA is an isothermal (constant temperature), nucleic acid amplification method. In SDA, displacement of single-stranded extension products, annealing of primers to the extension products (or the original target sequence) and subsequent extension of the primers occur concurrently in the reaction mix. Conventional SDA (performed at lower temperatures, usually about 35-45° C.) is described by G. Walker, et al., Proc. Natl. Acad. Sci. USA 89:392-396 (1992) and Walker et al., Nuc. Acids Res., supra. Detection of nucleic acids by SDA is described in detail in U.S. Pat. Nos. 5,455,166; 5,523,204; and 5,916,779, the entire teachings of which are herein incorporated by reference. These patents provide methods for amplification of a target nucleic acid sequence (and its complementary strand) in a sample by endonuclease-mediated strand displacement. Additionally, U.S. Pat. No. 5,916,779 adapts SDA to reverse transcription amplification of RNA targets.

According to the present invention, the SARS-CoV target nucleocapsid RNA is extracted from a test sample. The SARS-CoV nucleocapsid RNA may be isolated by any method known to those of skill in the art. The nucleocapsid RNA is then amplified in, for example, an RT-SDA process. The RT-SDA may be performed as either a one-step process or a two-step process. The one-step process concurrently generates and amplifies cDNA copies of the SARS-CoV target sequence.

In one embodiment, the one-step RT-SDA process utilizes first amplification and bumper primers designed to allow for incorporation of a restriction endonuclease site and for displacement of single stranded cDNA. The resulting cDNA is subsequently amplified by annealing of second amplification and optionally one or more bumper primers. In another embodiment, the one-step RT-SDA process utilizes a first reverse and optionally one or more bumper primers. Either DNA-dependent DNA polymerase or reverse transcriptase allows for the extension of the cDNA amplified products. In yet another embodiment of the single-step process, a reverse transcriptase enzyme is used to extend one or more of the reverse primers and synthesize cDNA from the RNA template. One of ordinary skill in the art will recognize certain conventional reverse transcriptase enzymes (i.e., AMV, MMLV, Superscript II™) that may be employed in the methods of the present invention.

The foregoing description of the one-step RT-SDA reaction uses SDA amplification primers and bumper primers as an illustrative example. As described in U.S. Pat. No. 5,916,779, however, the reverse transcriptase is capable of performing strand displacement with either SDA primers or reverse transcription primers. Reverse transcription primers may, therefore, also be present for use by the reverse transcriptase in the reverse transcription portion of the reaction. The downstream reverse transcription primer functions as a reverse transcription primer. The upstream reverse transcription primer is similar to an SDA bumper primer, as its extension serves to displace the downstream reverse transcription primer extension product (the cDNA).

Alternatively, the RT-SDA may be a two-step amplification process in which reverse transcription is followed by SDA in discrete steps. Accordingly, a reverse transcription primer is present in the first, reverse transcription step of the reaction. The cDNA is then separated from the RNA template prior to the second, amplification step. The reaction is either heated to separate the DNA:RNA hybrid, or the two strands are separated through chemical or enzymatic means. For example, but not by way of limitation, RNase H or RNase H activity may be used to degrade the RNA strand and thereby create a single strand of DNA. Also, separation of the hybrid can be achieved by the use of a polymerase that lacks 5′→3′ activity and displaces one strand from another. SDA primers are added in the second step of the reaction, and SDA amplification proceeds to provide detectable amplification products.

In one embodiment of the two-step process, the reverse primer is an SDA primer, and RNase H activity is endogenous to the reverse transcriptase enzyme. Additionally, the reverse primer may be a bumper primer or a randomly generated DNA sequence. In a further embodiment of the present invention, a two-step RT-SDA process is performed using an SDA primer and one or more bumper primers for the reverse transcription reaction. Forward primers and other reaction components necessary for amplification and detection, such as SDA enzymes, deoxyribonucleotides, signal primers, probe(s) and buffer components, are mixed with the products of the RT reaction.

A thermophilic version of the SDA reaction (tSDA) has recently been developed, and this version is performed at a higher, but still constant, temperature using thermostable polymerases and restriction endonucleases, as described in U.S. Pat. Nos. 5,648,211 and 5,744,311, which are incorporated by reference herein. The reaction is performed essentially as conventional SDA, with substitution of a thermostable polymerase and a thermostable restriction endonuclease. The temperature of the reaction is adjusted to a higher temperature suitable for the selected thermophilic enzymes (typically between about 45° C. and 60° C.), and the conventional restriction endonuclease recognition/cleavage site is replaced by the appropriate restriction endonuclease recognition/cleavage site for the selected thermostable endonuclease. Also, in contrast to conventional SDA, the practitioner may include the enzymes in the reaction mixture prior to the initial heat denaturation step if they are sufficiently stable at that temperature.

SDA has been adapted for amplification of nucleic acid target sequences in situ in cells in suspension, on slides or in tissues, with sensitivity and specificity comparable to in situ PCR. This method is described in detail in U.S. Pat. No. 5,523,204, which is incorporated herein by reference. SDA is gentler to the cells and tissues than is PCR because the SDA reaction is carried out at a constant, lower temperature. In addition, excellent specimen morphology is preserved. In situ amplification by SDA is compatible with immunochemical techniques, so that both amplification of target sequences and immunological staining can be performed on the same specimen.

An RNA-based internal control may be incorporated in the reaction mixture that co-amplifies with the SARS-CoV target sequences of the present invention. The internal control is designed to verify negative results and identify potentially inhibitory samples. Such a control may also be used for the purposes of quantification in a competitive assay format as described by Nadeau et al. Anal. Biochem. 276: 177-187 (1999). In addition, the use of dried Reverse Transcriptase enzyme may be used in conjunction with the SDA methods described herein. The dried enzyme provides improved workflow over use of liquid enzyme together with a protracted shelf life.

The SDA primers, Bumper Primers and Signal Primers listed in Table 1 and Table 3 were designed for use in RT-SDA reactions in accordance with the methods of the present invention. The binding sequences are underlined. For the SDA Primers, the remaining 5′ portion of the sequence comprises the restriction endonuclease recognition site (RERS) required for the SDA reaction to proceed and a generic non-target-specific tail sequence; whereas, for the Signal Primers, the 5′ tail comprises a generic non-target-specific sequence which is the same as that of the corresponding reporter probe. It will be readily apparent that the SDA primers may also be used as amplification primers in alternative amplification assays. It will also be apparent that the target binding sequences may be used alone to amplify the target in reactions that do not require specialized sequences or structures (e.g., PCR) and that different specialized sequences required by amplification reactions other than RT-SDA may be substituted for the RERS-containing sequence shown below (e.g., an RNA polymerase promoter). The “F” and “R” in the SDA primer name indicates “forward” and “reverse” primers, respectively, when the oligonucleotides are used in amplification reactions.

TABLE 1
Primers, Probes and Sequences for SARS-CoV Assay Region C

| | | | |
|---|---|---|---|
| SEQ ID | | | |
| NO. | Oligonucleotide | Length | 5′-3′ Sequence |

| | | | |
|---|---|---|---|
| | BUMPER PRIMERS | | |
| 1 | SARSrtB24 * | 24 | CAA TGT TGT TCC TTG AGG AAG |
| | | | TTG |
| 2 | SARSrtB21 * | 20 | GTT GTT CCT TGA GGA AGT TG |
| 3 | SARSrtB17 * | 17 | GTT CCT TGA GGA AGT TG |
| 24 | pUCl9 Bumper Primer | 15 | GCC TCT TCG CTA TTA |
| | CD | | |
| | SDA PRIMERS | | |
| 4 | SarCFP | 41 | CGA TTC CGC TCC AGA CTT CTC |
| | | | GGG AAC AAA GAA GGC ATC GT |
| 5 | SarCRP * | 41 | ACC GCA TCG AAT GCA TGT CTC |
| | | | GGG TGG GAG CAT TGT TAT TA |
| | SIGNAL PRIMERS | | |
| 6 | SarCAd-TBD16 | 40 | ACG TTA GCC ACC ATA CGG ATA |
| | | | CCC AAA GAC CAC ATT GGC A |
| 7 | SarG-IACAd | 40 | ACT GAT CCG CAC TAA CGA CTA |
| | | | CCC AAA GAC CAC ACG GAG T |
| 8 | SarCAd-MPC | 40 | ACG TTA GCC ACC ATA CTT GAA |
| | | | CCC AAA GAC CAC ATT GGC A |
| 25 | SarCIAC-Ad2 | 40 | AGC TAT CCG CCA TAA GCC ATA |
| | | | CCC AAA GAC CAC ACG GAC T |
| | TARGET | | |
| | SEQUENCES | | |
| 9 | Assay Region C | 126 | AAC AAA GAA GGC ATC GTA TGG |
| | Consensus | | GTT GCA ACT GAG GGA GCC TTG |
| | Cloned Target | | AAT ACA CCC AAA GAC CAC ATT |
| | Sequence | | GGC ACC CGC AAT CCT AAT TAC |
| | | | AAT GCT GCC ACC GTG CTA CAA |
| | | | CTT CCT CAA GGA ACA ACA TTG |
| 10 | Assay Region C | 126 | AAC AAA GAA GGC AUC GUA |
| | Consensus | | UGG GUU GCA ACU GAG GGA |
| | RNA Transcript | | GCC UUG AAU ACA CCC AAA GAG |
| | | | CAC AUU GGC ACC CGC AAU CCU |
| | | | AAU UAC AAU GCU GCC AGC GUG |
| | | | CUA CAA CUU CCU CAA GGA ACA |
| | | | ACA UUG |
| 11 | Assay Region C Cloned | 126 | AAC AAA GAA GGC ATC GTA TGG |
| | Internal Amplification | | GTT GCA ACT GAG GGA GCC TTG |
| | Control | | AAT ACA CCC AAA GAG GAG Acg |
| | | | gao tCC CGC AAT CCT AAT TAC |
| | | | AAT GCT GCC AGC GTG CTA CAA |
| | | | CTT CCT CAA GGA ACA ACA TTG |
| 12 | Assay Region C Internal | 126 | AAC AAA GAA GGC AUC GUA |
| | Amplification Control | | UGG GUU GCA ACU GAG GGA |
| | Transcript | | GCC UUG AAU ACA CCC AAA GAC |
| | | | CAG Acg gac uCC CGC AAU CCU |
| | | | AAU UAC AAU GCU GCC AGC GUG |
| | | | CUA CAA CUU CCU CAA GGA ACA |
| | | | ACA UUG |

Primer target hybridization regions are underlined.
BsoBI sites are italicized and shaded.
Mutations in the Internal Amplification Control relative to the native SARS-CoV consensus sequence are in lower case.
* Oligonucleotides that may be used to prime reverse transcription.

TABLE 2
Reporter Probes for use with SAPS-CoV Assays C and D

| | | | |
|---|---|---|---|
| SEQ ID | | | |
| NO. | Oligonucleotide | Length | 5′-3′ Sequence |

| | | | |
|---|---|---|---|
| | REPORTER PROBE | | |
| | SET A | | |
| 13 | TBD16 (D/R) | 28 | (Dabcyl)-TCC CGA GT-(Rox)-ACG |
| | | | TTA GCC ACC ATA CGG AT |
| 14 | AltD8(F/D) | 28 | (Fam)-ACC CGA GT-(Dabcyl)-AGC |
| | | | TAT CCG CCA TAA GCC AT |
| | REPORTER PROBE | | |
| | SET B | | |
| 15 | MPC(D/R) | 29 | (DABCYL)-TCC CCG AGT-(ROX)- |
| | | | ACG TTA GCC ACC ATA CTT GA |
| 16 | MPC2(F/D) | 29 | (FAM)-TCC CCG AGT-(DABCYL)- |
| | | | ACT GAT CCG CAC TAA CGA CT |

Regions that hybridize to the complement of the Signal Primers are underlined (see U.S. Pat. No. 6,316,200; 6,743,582; 6,656,680)
BsoBI sites are italicized.
ROX: Rhodamine.
FAM: Fluorescein.

TABLE 3
Primers, Probes and Sequences for SARS-CoV Assay Region D

| | | | |
|---|---|---|---|
| SEQ ID | | | |
| NO. | Oligonucleotide | Length | 5′-3′ Sequence |

| | | | |
|---|---|---|---|
| | BUMPER PRIMERS | | |
| 17 | SarDB22 * | 22 | ATG TTC CCG AAG GTG TGA CTT |
| | | | C |
| | SDA PRIMERS | | |
| 18 | SarDFP | 41 | CGA TTC CGC TCC AGA CTT CTC |
| | | | GGG ACA AGG AAC TGA TTA CA |
| 19 | SarDRP * | 41 | ACC GCA TCG AAT GCA TGT CTC |
| | | | GGG TGC GTG ACA TTC CAA AG |
| | SIGNAL PRIMERS | | |
| 20 | SarDAd-TBD16 | 40 | ACG TTA GCC ACC ATA CGG ATC |
| | | | AAT TTG CTC CAA GTG CCT C |
| 21 | SarDAd-MPC | 40 | ACG TTA GCC ACC ATA CTT GAC |
| | | | AAT TTG CTC CAA GTG CCT C |
| | TARGET | | |
| | SEQUENCES | | |
| 22 | ASSAY REGION D | 111 | GAC AAG GAA CTG ATT ACA AAC |
| | CONSENSUS | | ATT GGC CGC AAA TTG CAC AAT |
| | DNA Target Sequence | | TTG CTC CAA GTG CCT CTG CAT |
| | | | TCT TTG GAA TGT CAC GCA TTG |
| | | | GCA TGG AAG TCA CAC CTT CGG |
| | | | GAA CAT |
| 23 | ASSAY REGION D | 111 | GAC AAG GAA CUG AUU ACA |
| | CONSENSUS | | AAC AUU GGC CGC AAA UUG CAC |
| | RNA Transcript | | AAU UUG CUC CAA GUG CCU CUG |
| | | | CAU UCU UUG GAA UGU CAC GCA |
| | | | UUG GCA UGG AAG UCA CAC CUU |
| | | | CGG GAA CAU |

Primer target hybridization regions are underlined.
BsoBI sites are italicized.
* Ollgonucleotides that may be used to prime reverse transcription.

Following target amplification, the nucleic acids produced by the methods of the present invention may be detected by any of the methods known in the art for detection of specific nucleic acid sequences. For example, but not by way of limitation, a variety of detection methods for SDA may be used. Several methods for labeling SDA products are discussed in U.S. Pat. No. 6,316,200, the entire teaching of which is herein incorporated by reference. For example, but not by way of limitation, amplification products may be detected by specific hybridization to an oligonucleotide detector probe. The detector probe is a short oligonucleotide that includes a detectable label, i.e., a moiety that generates or can be made to generate a detectable signal. The label may be incorporated into the oligonucleotide probe by nick translation, end-labeling or during chemical synthesis of the probe. Many directly and indirectly detectable labels are known in the art for use with oligonucleotide probes. Directly detectable labels include those labels that do not require further reaction to be made detectable, e.g., radioisotopes, fluorescent moieties and dyes. Indirectly detectable labels include those labels that must be reacted with additional reagents to be made detectable, e.g., enzymes capable of producing a colored reaction product (e.g., alkaline phosphatase (AP) or horseradish peroxidase), biotin, avidin, digoxigenin (dig), antigens, haptens or fluorochromes. The signal from enzyme labels is generally developed by reacting the enzyme with its substrate and any additional reagents required to generate a colored enzymatic reaction product. Biotin (or avidin) labels may be detected by binding to labeled avidin (or labeled biotin) or labeled anti-biotin (or labeled anti-avidin) antibodies. Digoxigenin and hapten labels are usually detected by specific binding to a labeled anti-digoxigenin (anti-dig) or anti-hapten antibody. In general, the detector probe will be selected such that it hybridizes to a nucleotide sequence in the amplicon that is between the binding sites of the two amplification primers. A detector probe may also have the same nucleotide sequence as either of the amplification primers. Methods for detection in vitro and in situ by hybridization to a detector probe are known in the art.

Alternatively, the amplification products of the present invention may be detected by extension of a detector primer as described by Walker, et al., Nuc. Acids Res., supra. In the detector primer extension method, an oligonucleotide primer comprising a detectable label is hybridized to the amplification products and extended by addition of polymerase. For detection, the primer may be 5′ end-labeled, for example, using ³²P or a fluorescent label. Alternatively, extension of the hybridized primer may incorporate a dNTP analog comprising a directly or indirectly detectable label. For example, but not by way of limitation, extension of the primer may incorporate a dig-derivatized dNTP, which is then detected after extension by reaction with AP anti-dig and a suitable AP substrate. The primer to be extended may either be the same as an amplification primer or it may be a different primer that hybridizes to a nucleotide sequence in the amplicon that is between the binding sites of the amplification primers.

The detectable label may also be incorporated directly into amplicons during target sequence amplification.

In another embodiment of the invention, RT-SDA products are detected by the methods described in U.S. Pat. No. 6,316,200 that utilize an unlabelled signal primer comprising a 5′ adapter sequence. The 3′ end of a reporter probe hybridizes to the complement of the 5′ end of the signal primer, producing a 5′ overhang. Polymerase fills in the overhang and synthesis of the complement of the reporter probe tail is detected, either directly or indirectly, as an indication of the presence of target. This method utilizes fluorescent energy transfer (FET) rather than the direct detection of fluorescent intensity for detection of hybridization. FET allows for real-time detection of SDA products.

The Signal Primers and Reporter Probes in Table 1 through Table 3 are designed for real-time detection of amplification products using the reverse transcriptase products. The structure and use of such primers and probes is described, for example, but not by way of limitation, in U.S. Pat. Nos. 5,547,861, 5,928,869, 6,316,200, 6,656,680 and 6,743,582 each of which is incorporated herein by reference. The hybridization sequences in Tables 1 through Table 3 are underlined. The remaining portions of the Reporter Probe sequences form structures that are typically labeled to facilitate detection of amplification products as is known in the art. It will be readily apparent that the target sequence may be used alone for direct hybridization (typically linked to a detectable label) and that other directly and indirectly labels may be substituted for the hairpin as is known in U.S. Pat. Nos. 5,935,791; 5,846,726; 5,691,145; 5,550,025; and 5,593,867, the contents of each of which is incorporated herein by reference.

Because the target binding sequence confers target specificity on the primer or probe, it should be understood that the target binding sequences exemplified above for use as particular components of a specified reaction may also be used in a variety of other ways for the detection of SARS-CoV nucleocapsid nucleic acid. For example, but not by way of limitation, the target binding sequences of the invention may be used as hybridization probes for direct detection of SARS-CoV, either without amplification or as a post-amplification assay. Such hybridization methods are well-known in the art and typically employ a detectable label associated with or linked to the target binding sequence to facilitate detection of hybridization. Further, essentially all of the target binding sequences set forth above may be used as amplification primers in amplification reaction which do not require additional specialized sequences (such as PCR) or appended to the appropriate specialized for use in 3SR, NASBA, transcription-based or any other primer extension amplification reactions. For detection of amplification products, amplification primers comprising the target binding sequences disclosed herein may be labeled as is known in the art. As an alternative, labeled detector primers comprising the disclosed target binding sequences may be used in conjunction with amplification primers as described in U.S. Pat. Nos. 5,547,861; 5,928,869; 5,593,867; 5,550,025; 5,935,791; 5,888,739; and 5,846,726, each of which is incorporated by reference herein, for real-time homogenous detection of amplification. Such detector primers may comprise a directly or indirectly detectable sequence that does not initially hybridize to the target but which facilitates detection of the detector primer once it has hybridized to the target and has been extended. For example, such detectable sequences may be sequences that form a secondary structure, sequences that contain a restriction site, or linear sequences that are detected by hybridization of their complements to a labeled oligonucleotide (sometimes referred to as a reporter probe) as is known in the art. Alternatively, the amplification products may be detected post-amplification by hybridization of a probe selected from any of the target binding sequences disclosed herein that fall between a selected set of amplification primers.

It is to be understood that an oligonucleotide according to the present invention that consists of a target binding sequence and, optionally, either a sequence required for a selected amplification reaction or a sequence required for a selected detection reaction may also include certain other sequences that serve as spacers, linkers, sequences for labeling or binding of an enzyme, etc. Such additional sequences are typically known to be necessary to obtain optimum function of the oligonucleotide in the selected reaction and are intended to be included by the term “consisting of.”

The present invention also relates to nucleic acid molecules that hybridize under differing stringency hybridization conditions (i.e., for selective hybridization) to the nucleotide sequence described herein. “Stringency conditions” refer to the incubation and wash conditions (e.g., temperature, buffer concentration) that determine hybridization of a first nucleic acid to a second nucleic acid. The first and second nucleic acids may be perfectly (100%) complementary, or may be less than perfect (i.e., 70%, 50%, etc.). For example, certain high stringency conditions can be used that distinguish perfectly complementary nucleic acids from those of less complementarity. “High stringency conditions,” “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., John Wiley & Sons (1998), the entire teachings of which are incorporated by reference herein).

Another aspect of the present invention pertains to host cells into which a vector of the invention has been introduced. A host cell can be any prokaryotic or eukaryotic cell. For example, the nucleic acid molecules of the present invention can be expressed in bacterial cells, insect cells, yeast or mammalian cells. Such suitable host cells are known to those skilled in the art.

The invention also provides a pack or kit comprising one or more containers filled with one or more of the ingredients used in the present invention. Optionally associated with such container(s) can be a notice in the form prescribed by a governmental agency regulating the manufacture, use or sale of pharmaceuticals or biological products, which notice reflects approval by the agency for manufacture, use or sale for administration. The pack or kit can be a single unit use of the compositions or it can be a plurality of uses. In particular, the agents can be separated, mixed together in any combination, or present in a single vial.

The following prophetic examples are provided to illustrate certain embodiments of invention, but are not intended to limit the invention.

### SARS System D Examples: RT-SDA for the Detection of SARS-CoV RNA

The following example illustrates the use of the disclosed primers and reporter Probes for the detection of SARS-CoV RNA in clinical specimens.

Clinical specimens such as stool samples, throat swabs and nasopharyngeal aspirates are processed using a QIAGEN QIAamp Viral RNA Mini Kit according to the manufacturer's instructions with the addition of an on-column DNase treatment to remove contaminating DNA. For stool specimens, an additional pre-processing step is included to remove particulate matter prior to loading on the QIAGEN columns. Stools are diluted 1:10 with 0.89% saline and centrifuged for 20 min. at 4,000×g. The supernatant is then decanted and passed through a 0.22 μm filter to remove particulate debris.

One hundred and forty microliters of the sample or stool filtrate are processed through a QIAamp column that is treated with DNase to digest contaminating non-specific DNA bound to the column matrix. After washing to remove the DNase, purified RNA is eluted in a volume of 80 μL water. Thirty microliters of eluate are added to a Priming Microwell containing dried primers, Reporter Probes and nucleotides, followed by 20 μL of Reverse Transcription Buffer containing RNase inhibitor, AMV-RT enzyme and RNA transcripts of an RNA-based IAC sequence. Final reaction conditions for reverse transcription are as follows: 1500 μM dCₛTP; 300 μM each of dATP, dGTP and dTTP; 5 mM magnesium acetate; 1500 nM bumper primer SarDB22 (SEQ ID NO.: 17); 1500 nM SDA Primer SarDRP (SEQ ID NO.: 19); 300 nM SDA Primer SarDFP (SEQ ID NO.: 18); 750 nM Signal Primer SarDAd-MPC (SEQ ID NO.: 21); 600 nM IAC Signal Primer; 1200 nM Reporter Probe MPC D/R (SEQ ID NO.: 15); 900 nM Reporter Probe MPC2 F/D (SEQ ID NO.: 16); approximately 1000 copies of IAC transcript; 5% DMSO; 5% glycerol; 43.5 mM KᵢPO₄; 25 mM KOH; 120 mM bicine; 40U RNase inhibitor; 10U AMV-RT. Rehydrated microwells are then incubated at 48° C. for 20 min. before addition of 100 μL of SDA Buffer and transfer to a 72° C. heat block. At the same time, Amplification Microwells containing dried SDA enzymes (Bst polymerase and BsoBI restriction enzyme) are pre-warmed at 54° C. After a 10 min incubation, 100 μL of sample are transferred from the Priming Microwells to the Amplification Microwells, which are then sealed and incubated in a BD ProbeTec ET reader at 52.5° C. Final reaction conditions for SDA are as follows: 500 μM dCsTP; 100 μM each of dATP, dGTP and dTTP; 5.7 mM magnesium acetate; 500 nM Bumper Primer SarDB22 (SEQ ID NO.: 17); 500 nM SDA Primer SarDRP (SEQ ID NO.: 19); 100 nM SDA Primer SarDFP (SEQ ID NO.: 18); 250 nM Signal Primer SarDAd-MPC (SEQ ID NO.: 21); 200 nM IAC Signal Primer; 400 nM target Reporter Probe MPC D/R (SEQ ID NO.: 15); 300 nM IAC Reporter Probe MPC2 F/D (SEQ ID NO.: 16); 12.5% DMSO; 1.67% glycerol; 24.5 mM KᵢPO₄; 82 mM KOH; 143 mM bicine; 12U Bst polymerase; 45U BsoBI restriction enzyme.

During the course of a 1 hour incubation, fluorescent readings are taken every minute in both optical channels of the BD ProbeTec ET instrument and results are reported in terms of the PAT scores for the SARS-CoV target and IAC. Reactions in which the fluorescent readings never achieve the predetermined threshold of fluorescence are assigned a PAT score of 0. Reactions that yielded ROX PAT scores >0, corresponding to the MPC D/R Reporter Probe (SEQ ID NO.: 15), are considered positive for SARS-CoV, while reactions that yield FAM PAT scores >0, corresponding to the IAC Reporter Probe MPC2 F/D (SEQ ID NO.: 16), are considered positive for IAC. Those in which neither the FAM nor ROX signals achieve their respective thresholds (PAT scores=0) are considered indeterminate. External positive and negative controls are included in each assay run to verify performance. These controls are required to yield the positive and negative correct results respectively in order for the results from patient specimens to be reported by the instrument.

### Anticipated Results and Conclusions

Specimens from infected patients that contain SARS-CoV in sufficient quantity to be above the limit of detection of the assay would yield positive results (i.e., ROX PAT scores >0). Specimens from uninfected patients or from those whose clinical load is below the analytical sensitivity of the assay would yield negative results (i.e., ROX PAT score=0). Contamination of reagents with RNase or procedural error would be indicated by a failure of the IAC to amplify (i.e., FAM PAT score=0). A summary of possible results is presented in Table 4.

TABLE 4
Summary of possible result outcomes
for the BD ProbeTec ET SARS-CoV assay

| PAT Score | | |
|---|---|---|
| SARS-CoV Target | | |
| (ROX) | IAC (FAM) | Reported Result |
| >0 | Any | Positive for SARS-CoV |
| 0 | 0 | Indeterminate |
| 0 | >0 | Negative for SARS-CoV or |
| | | virus present below the |
| | | analytical sensitivity |
| | | of the assay |

The following experimental examples are provided to illustrate certain embodiments of the invention, but are not intended to limit the invention.

### SARS Assay System C Examples

### Example 1

### DNA Amplification Using SARS-CoV-Specific Primers

The ability of the disclosed combination of primers and probes to amplify SARS-CoV nucleic acid was demonstrated using a pUC19-based plasmid clone of the target region (corresponding to nucleotides 28494-28619 of SARS-CoV strain BJ03; GenBank Accession No. AY278490). Plasmid DNA was linearized with the restriction enzyme Nan and quantified using PicoGreen® dsDNA Quantitation Reagent (Molecular Probes, Inc., Eugene, Oreg.). Prior to amplification, the quantified plasmid stock was diluted to a working concentration of 25 copies/A in 10 ng/A salmon sperm DNA. Four replicate SDA reactions were run at each of six target levels, in addition to control reactions containing water in place of target DNA.

In brief, DNA target was added to SDA Buffer and denatured by heating in a boiling water bath for 5 min. One hundred and fifty microliters of the denatured sample was then added to Priming Microwells containing dried SDA Primers, Reporter Probes and nucleotides. After 20 min. at ambient temperature, the Priming Microwells were transferred to a heat block at 72° C., while corresponding Amplification Microwells containing dried Bst polymerase and BsoBI restriction enzyme were pre-warmed at 54° C. Following a 10 min. incubation, 100 μL of the priming mixture were transferred from the Priming to the Amplification Microwells, which were then sealed and placed at 52.5° C. in a BD ProbeTec ET reader. Fluorescent signals, were monitored over the course of 1 hour and analyzed using the Passes After Threshold (PAT) algorithm developed for this instrument. Wolfe D M, Wang S S, Thornton K, Kuhn A M, Nadeau J G, Hellyer T J. Homogeneous strand displacement amplification. In DNA amplification—current technologies and applications, Demidov V V and Broude N E (Eds.), Horizon Bioscience, Wymondham, UK. The PAT scores represent the number of instrument passes remaining after the fluorescent readings achieve a pre-defined threshold value. Final SDA reaction conditions were as follows: 50 nM pUC19-based Bumper Primer CD (SEQ ID NO.: 24); 500 nM SDA Primer SarCRP (SEQ ID NO.: 5); 100 nM SDA Primer SarCFP (SEQ ID NO.: 4); 250 nM Signal Primer SarCAd-MPC (SEQ ID NO.: 8); 300 nM Reporter Probe MPC D/R (SEQ ID NO.: 15); 500 μM deoxycytidine 5′-O-(1-Thiotriphosphate), S-isomer (dCₛTP); 100 μM each of dATP, dGTP and dTTP; 12.5% DMSO; 24.5 mM KᵢPO₄; 82 mM KOH; 143 mM bicine; 12U Bst polymerase; 30U BsoBI restriction enzyme; 5 mM magnesium acetate.

### Results and Conclusions

Positive results were obtained with as little 6 copies of the target plasmid per reaction while no false-positive results were observed in any of the negative controls (Table 5). These data demonstrate that the disclosed combination of primers and Reporter Probe is capable of detecting a SARS-CoV-specific nucleic acid target sequence with a high degree of analytical sensitivity.

TABLE 5
Amplification and detection of a
SARS-CoV-specific target sequence

| Target Level | PAT Score | | | | |
|---|---|---|---|---|---|
| Per Reaction | A | B | C | D | Mean |
| 100 | 48.1 | 49.1 | 46.8 | 47.7 | 47.9 |
| 50 | 45.7 | 46.2 | 45.5 | 45.2 | 45.7 |
| 25 | 44.1 | 47.9 | 44.8 | 44.5 | 45.3 |
| 12.5 | 0 | 48.8 | 44.3 | 45.6 | 34.7 |
| 6.25 | 35.3 | 0 | 45.4 | 0 | 20.2 |
| 3.13 | 0 | 0 | 0 | 0 | 0 |
| 1.56 | 0 | 0 | 0 | 0 | 0 |
| 0 | 0 | 0 | 0 | 0 | 0 |

PAT scores: >0 = Positive; 0 = Negative

### Example 2

### Analytical Specificity

### Part A:

The analytical specificity of the disclosed primers and probes was verified by testing a panel of 51 bacteria and fungi that are likely to be found in respiratory and/or gastrointestinal specimens (Table 5). Because all these organisms have genomes comprised of DNA rather than RNA, no reverse transcription step was included in these reactions. A suspension of each organism was prepared in Phosphate-Buffered Saline containing Bovine Serum Albumin (PBS/BSA) at a concentration of approximately 10⁷-10⁸ cells/mL. Fifteen microliters of each suspension were mixed with 150 μL SDA Buffer and heated in a boiling water bath for 5 min. After cooling to room temperature, 150 μL of denatured sample were added to a Priming Microwell containing dried SDA Primers, Reporter Probe and nucleotides. The Priming Microwells were incubated at ambient temperature for 20 min. and then transferred to a heat block at 72° C., while corresponding Amplification Microwells were pre-warmed at 54° C. After a 10 min. incubation, 100 μL of the priming mixture were transferred from the Priming to the Amplification Microwells, which were then sealed and loaded into a BD ProbeTec ET reader set at 52.5° C. Fluorescence was monitored over the course of 1 hour and analyzed using the PAT algorithm developed for this instrument. Final SDA reaction conditions were as follows: 50 nM pUC19 Bumper Primer CD (SEQ ID NO.: 24); 500 nM SDA Primer SarCRP (SEQ ID NO.: 5); 100 nM SDA Primer SarCFP (SEQ ID NO.: 4); 250 nM Signal Primer SarCAd-MPC (SEQ ID NO.: 8); 300 nM Reporter Probe MPC D/R (SEQ ID NO.: 15); 500 μM dCₛTP; 100 μM each of dATP, dGTP and dTTP; 12.5% DMSO; 24.5 mM KᵢPO₄; 82 mM KOH; 143 mM bicine; 12U Bst polymerase; 30U BsoBI restriction enzyme; 5 mM magnesium acetate.

### Results and Conclusions

No positive results were obtained except from a plasmid clone of the SARS-CoV target sequence that was run as a positive control, thereby demonstrating the specificity of the disclosed primers and Reporter Probe for the detection of SARS-CoV. Results are shown in Table 6.

TABLE 6
Panel of bacteria and fungi tested with
the BD ProbeTec ET SARS-CoV assay

| | | PAT | |
|---|---|---|---|
| Species | Strain | Score | Result |
| Acinetobacter calcoaceticus | BD 13339 | 0 | Negative |
| Actinomyces israelii | ATCC 10049 | 0 | Negative |
| Aeromonas hydrophila | ATCC 7966 | 0 | Negative |
| Alcaligenes faecalis | ATCC 8750 | 0 | Negative |
| Bacteroides fragilis | ATCC 25285 | 0 | Negative |
| Blastomyces dermatitidis | ATCC 4292 | 0 | Negative |
| Bordetella pertussis | ATCC 9797 | 0 | Negative |
| Branhamella catarrhalis | ATCC 25238 | 0 | Negative |
| Candida albicans | ATCC 44808 | 0 | Negative |
| Chlamydophila pneumoniae | AR-39 | 0 | Negative |
| Citrobacter freundii | ATCC 8090 | 0 | Negative |
| Clostridium perfringens | ATCC 13124 | 0 | Negative |
| Corynebacterium diphtheriae | ATCC 11913 | 0 | Negative |
| Corynebacterium jeikeium | ATCC 43734 | 0 | Negative |
| Cryptococcus neoformans | ATCC 36556 | 0 | Negative |
| Edwardsiella tarda | ATCC 15469 | 0 | Negative |
| Eikenella corrodens | ATCC 23834 | 0 | Negative |
| Enterobacter aerogenes | ATCC 13048 | 0 | Negative |
| Enterococcus faecalis | ATCC 29212 | 0 | Negative |
| Escherichia coli | ATCC 11775 | 0 | Negative |
| Fusobacterium nucleatum | ATCC 25586 | 0 | Negative |
| Haemophilus influenzae | ATCC 33533 | 0 | Negative |
| Haemophilus parainfluenzae | ATCC 7901 | 0 | Negative |
| Histoplasma capsulatum | ATCC 12700 | 0 | Negative |
| Kingella kingae | ATCC 23330 | 0 | Negative |
| Klebsiella pneumoniae subsp. | ATCC 13883 | 0 | Negative |
| pneumoniae | | | |
| Lactobacillus acidophilus | ATCC 4356 | 0 | Negative |
| Legionella pneumophila | ATCC 33152 | 0 | Negative |
| Moraxella osloensis | ATCC 19976 | 0 | Negative |
| Morganella morganii | ATCC 25830 | 0 | Negative |
| Mycobacterium tuberculosis | ATCC 27294 | 0 | Negative |
| Mycoplasma pneumoniae | ATCC 29342 | 0 | Negative |
| Neisseria meningitides | ATCC 13077 | 0 | Negative |
| Neisseria mucosa | ATCC 19696 | 0 | Negative |
| Peptostreptococcus anaerobius | ATCC 27337 | 0 | Negative |
| Plesiomonas shigelloides | ATCC 14029 | 0 | Negative |
| Porphyromonas asaccharolytica | ATCC 25260 | 0 | Negative |
| Proteus mirabilis | ATCC 29906 | 0 | Negative |
| Providencia stuartii | ATCC 35031 | 0 | Negative |
| Pseudomonas aeruginosa | ATCC 27853 | 0 | Negative |
| Serratia marcescens | ATCC 8100 | 0 | Negative |
| Staphylococcus aureus | ATCC 12598 | 0 | Negative |
| Staphylococcus epidermidis | ATCC E155 | 0 | Negative |
| Stenotrophomonas maltophila | ATCC 13637 | 0 | Negative |
| Streptococcus mitis | ATCC 6249 | 0 | Negative |
| Streptococcus mutans | ATCC 25175 | 0 | Negative |
| Streptococcus pneumoniae | ATCC 6303 | 0 | Negative |
| Streptococcus pyogenes | ATCC 19615 | 0 | Negative |
| Veillonella parvula | ATCC 10790 | 0 | Negative |
| Vibrio parahaemolyticus | ATCC 17802 | 0 | Negative |
| Yersinia enterolitica | ATCC 27729 | 0 | Negative |
| SARS-CoV Positive Control | Not | 41.4 | Positive |
| | Applicable | | |
| SARS-CoV Positive Control | Not | 43.5 | Positive |
| | Applicable | | |
| SARS-CoV Negative Control | Not | 0 | Negative |
| | Applicable | | |
| SARS-CoV Negative Control | Not | 0 | Negative |
| | Applicable | | |

BD: BD Diagnostics
ATCC: American Type Culture Collection
PAT scores >0 were considered positive

### Part B:

A second experiment was conducted with three non-SARS-related strains of coronavirus (non-SARS-CoV) using the complete RT-SDA assay format. Stock vials of non-SARS-CoV were obtained from the American Type Culture Collection and in diluted in PBS/BSA. One hundred and forty microliters of viral suspension were processed using a modified QIAGEN QIAamp Viral RNA Mini Kit procedure that incorporated an on-column DNase treatment to remove contaminating DNA. A suspension containing 400 particles of SARS-CoV was processed in parallel as a positive control. A second positive control was also included that contained in vitro transcripts derived from a plasmid clone of the SARS-CoV target sequence (SEQ ID NOs.: 9 and 10). RT-SDA was performed by pipetting 304 processed specimen into Priming Microwells containing dried SDA Primers, Reporter Probes and nucleotides, followed by 20 μL Reverse Transcription Buffer containing RNase inhibitor and Avian Myelobastosis Virus Reverse Transcriptase (AMV-RT). The microwells containing the reverse transcription reactions were then incubated at 48° C. for 20 min. The final reaction conditions were as follows: 120 mM bicine; 25 mM KOH; 43.5 mM KᵢPO₄; 5% DMSO; 5% glycerol; 1500 μM dC₂TP; 300 μM each of dATP, dGTP and dTTP; 5 mM magnesium acetate; 1500 nM Bumper Primer SARSrtB24 (SEQ ID NO.: 1); 1500 nM SDA Primer SarCRP (SEQ ID NO.: 5); 300 nM SDA Primer SarCFP (SEQ ID NO.: 4); 750 nM Signal Primer SarCAd-MPC (SEQ ID NO.: 8); 600 nM Internal Amplification Control (IAC) Signal Primer SarC-IACAd (SEQ ID NO.: 7); 1200 nM target Reporter Probe MPC D/R (SEQ ID NO.: 15); 900 nM IAC Reporter Probe MPC2 F/D (SEQ ID NO.: 16); 40U RNase inhibitor and 10U AMV-RT (both Promega Corp., Madison, Wis.).

At the end of the reverse transcription reaction, 100 μL SDA Buffer were added to each microwell, which were then transferred to a 72° C. heat block. At the same time, Amplification Microwells containing dried SDA enzymes (Bst polymerase and BsoBI restriction enzyme) were pre-warmed at 54° C. After a 10 min. incubation, 100 μL of sample were transferred from the Priming Microwells to the Amplification Microwells, which were then sealed and placed in a BD ProbeTec ET reader set at 52.5° C. Fluorescence was monitored over a 1 hour period and readings were analyzed using the PAT algorithm that was developed for the BD ProbeTec ET system. SDA conditions in the final reaction mixtures were as follows: 500 μM dCₛTP; 100 μM each of dATP, dGTP and dTTP; 5.7 mM magnesium acetate; 500 nM Bumper Primer SARSrtB24 (SEQ ID NO.: 1); 500 nM SDA Primer SarCRP (SEQ ID NO.: 5); 100 nM SDA Primer SarCFP (SEQ ID NO.: 4); 250 nM Signal Primer SarCAd-MPC (SEQ ID NO.: 8); 200 nM IAC Signal Primer SarC-IACAd (SEQ ID NO.: 7); 400 nM target Reporter Probe MPC D/R (SEQ ID NO.: 15); 300 nM IAC Reporter Probe MPC2 F/D (SEQ ID NO.: 16); 12.5% DMSO; 1.7% glycerol; 24.5 mM KᵢPO₄; 82 mM KOH; 143 mM bicine; 12U Bst polymerase; 45U BsoBI restriction enzyme.

### Results and Conclusions

Results are summarized in Table 7. As with the DNA amplification system in the previous experiment, no positive results were obtained except from reactions containing SARS-CoV target nucleic acid, thereby demonstrating the specificity of the disclosed primers and Reporter Probes for this analyte. No IAC target RNA was included in these reactions and therefore no signal above background was detected from the MPC2 F/D Reporter Probe (data not shown).

TABLE 7
Panel of non-SARS-CoV tested with
the BD ProbeTec ET SARS-CoV assay

| | | PAT Score | | | |
|---|---|---|---|---|---|
| Sample | Target Level | A | B | Mean | Result |
| CoV Strain E229 | Undiluted | 0 | 0 | 0 | Negative |
| CoV Strain OC43 | Undiluted | 0 | 0 | 0 | Negative |
| CoV Strain Dallas-1 | Undiluted | 0 | 0 | 0 | Negative |
| CoV Strain E229 | 1:100 of stock | 0 | 0 | 0 | Negative |
| CoV Strain OC43 | 1:100 of stock | 0 | 0 | 0 | Negative |
| CoV Strain Dallas-1 | 1:100 of stock | 0 | 0 | 0 | Negative |
| SARS-CoV | 150 particles | 52.7 | 52.3 | 52.5 | Positive |
| SARS-CoV | 300 particles | 0 | 0 | 0 | Negative |
| w/o AMV-RT | | | | | |
| SARS-CoV | 300 copies | 50.5 | 46.5 | 49.5 | Positive |
| transcript * | | | | | |
| Negative Control | Not applicable | 0 | 0 | 0 | Negative |

* Derived in vitro from a plasmid clone of the SARS-CoV target region (SEQ ID NOs.: 9 and 10)
PAT scores >0 were considered positive.

### Example 3

### Analytical Limit of Detection

The analytical limit of detection (LOD) of the BD ProbeTec ET SARS-CoV assay was determined by testing in vitro transcripts of the SARS-CoV target sequence (SEQ ID NOs.: 9 and 10). Transcripts were generated from a pUC19-based plasmid containing a copy of the SARS-CoV target that was inserted into the multiple cloning site downstream of an SP6 RNA polymerase promoter. Purified transcripts were diluted in 10 ng/μL yeast RNA as a carrier and a total of 24 assay replicates were tested at each of six target levels. In brief, 304 diluted target were added to Priming Microwells containing dried primers, Reporter Probes and nucleotides, followed by 20 μL of Reverse Transcription Buffer containing RNase inhibitor, AMV-RT and 1000 copies of RNA-based IAC (SEQ ID NO.: 12). Conditions for reverse transcription were as follows: 1500 μM dCₛTP; 300 μM each of dATP, dGTP and dTTP; 5 mM magnesium acetate; 1500 nM Bumper Primer SARSrtB24 (SEQ ID NO.: 1); 1500 nM SDA Primer SarCRP (SEQ ID NO.: 5); 300 nM SDA Primer SarCFP (SEQ ID NO.: 4); 750 nM Signal Primer SarCAd-MPC (SEQ ID NO.: 8); 600 nM IAC Signal Primer SarC-IACAd (SEQ ID NO.: 7); 1200 nM target Reporter Probe MPC D/R (SEQ ID NO.: 15); 900 nM IAC Reporter Probe MPC2 F/D (SEQ ID NO.: 16); 1000 copies of IAC transcript (SEQ ID NO.: 12); 5% DMSO; 5% glycerol; 43.5 mM KᵢPO₄; 25 mM KOH; 120 mM bicine; 40U RNase inhibitor; 10U AMV-RT.

Rehydrated microwells were incubated at 48° C. for 20 min before addition of 100 μL of SDA Buffer and transfer to a 72° C. heat block. At the same time, microwells containing dried SDA enzymes (Bst polymerase and BsoBI restriction enzyme) were pre-warmed on a heat block at 54° C. After a 10 min. incubation, 100 μL of sample were transferred from the Priming Microwells to the Amplification Microwells, which were then sealed and loaded into a BD ProbeTec ET reader set at 52.5° C. Final SDA reaction conditions were as follows: 500 μM dCₛTP; 100 μM each of dATP, dGTP and dTTP; 5.7 mM magnesium acetate; 500 nM Bumper Primer SARSrtB24 (SEQ ID NO.: 1); 500 nM SDA Primer SarCRP (SEQ ID NO.: 5); 100 nM SDA Primer SarCFP (SEQ ID NO.: 4); 250 nM Signal Primer SarCAd-MPC (SEQ ID NO.: 8); 200 nM IAC Signal Primer SarC-IACAd (SEQ ID NO.: 7); 400 nM Reporter Probe MPC D/R (SEQ ID NO.: 15); 300 nM IAC Reporter Probe MPC2 F/D (SEQ ID NO.: 16); 12.5% DMSO; 1.7% glycerol; 24.5 mM KᵢPO₄; 82 mM KOH; 143 mM bicine; 12U Bst polymerase; 45U BsoBI restriction enzyme.

During the course of a 1 hour incubation, fluorescent readings were taken every minute in both optical channels of the BD ProbeTec ET instrument and results were reported in terms of the PAT scores for the SARS-CoV target and IAC. Reactions in which the fluorescent readings never achieved the predetermined threshold of fluorescence were assigned a PAT score of 0. Reactions that yielded ROX PAT scores >0, corresponding to the MPC D/R Reporter Probe (SEQ ID NO.: 15), were considered positive for SARS-CoV, while reactions that yielded FAM PAT scores >0, corresponding to the IAC Reporter Probe MPC2 F/D (SEQ ID NO.: 16), were considered positive for IAC. Those in which neither the FAM nor ROX signals achieved their respective thresholds (PAT scores=0) were considered indeterminate. External positive and negative controls were included in each assay run to verify performance. These controls were required to yield the positive and negative correct results respectively in order for the results from patient specimens to be reported by the instrument.

A similar LOD study was conducted using Armored RNA® particles (Ambion, Inc., Austin, Tex.) consisting of a cloned copy of the SARS-CoV target RNA sequence, packaged in a nuclease resistant coliphage protein coat. Briefly, Armored RNA particles were diluted in TSMG buffer (10 mM Tris-HCl, pH7.0; 100 mM NaCl; 1 mM MgCl₂; 0.1% gelatin) and processed using a QIAGEN QIAamp Viral RNA Mini Kit. To mimic the treatment of a clinical specimen, an on-column DNase treatment step was incorporated to remove contaminating DNA from the sample. Purified RNA was eluted in a volume of 804 water and the assay was conducted as described above using 30 μL eluted sample. Fluorescent readings were collected over the course of 1 hour using a BD ProbeTec ET instrument and results were analyzed with the PAT algorithm.

### Results and Conclusions

The analytical sensitivity of the BD ProbeTec ET SARS-CoV assay was determined to be 201 copies of in vitro transcript or 149 copies of Armored RNA per reaction (Table 8). No indeterminate results were observed although one unspiked sample in the Armored RNA experiment yielded a positive result, probably due to cross-contamination of the sample during specimen processing.

TABLE 8
Analytical LOD of the BD ProbeTec ET SARS-CoV RT-SDA assay
for in vitro transcripts and Armored RNA particles

| | |
|---|---|
| In vitro transcripts | Armored RNA |

| Targets Per | | Targets Per | |
|---|---|---|---|
| Reverse | | Reverse | |
| Transcription | % Positive ^{a} | Transcription | % Positive ^{a} |
| Reaction | (n = 24) | Reaction | (n = 16) |
| 0 | 0 | 0 | 6.3 ^{c} |
| 50 | 54.2 | 31 | 43.8 |
| 100 | 70.8 | 63 | 68.8 |
| 200 | 96.8 | 125 | 87.5 |
| 400 | 100 | 250 | 100 |
| 500 | 100 | 500 | 100 |
| 95% LOD ^{b} | 201 | 95% LOD ^{b}** | 149 |
| | (114, 287) | | (131, 167) |

^{a} Positive result: PAT score >0
^{b} Lowest level detectable 95% of the time (95% confidence intervals)
^{c} One false-positive result probably due to laboratory cross-contamination during specimen processing

### Example 4

### Clinical Performance

A semi-optimized RT-SDA assay for SARS-CoV was evaluated in a clinical study performed in the Department of Microbiology at Queen Mary Hospital in Hong Kong using retrospective specimens obtained during the SARS outbreak in the winter of 2002-2003.

The specific objectives of this study were as follows:
1. To estimate the feasibility of the BD ProbeTec ET SARS-CoV assay for detection of SARS-CoV with respect to:
   Centers for Disease Control and Prevention (CDC) and/or World Health Organization (WHO) Case Definition (Combined case definition and separated Probable and Suspect case definitions for reference positive patients) (WHO. 2003. Case definitions for surveillance of Severe Acute Respiratory Syndrome (SARS): http://www.who.int/csr/sars/casedefinition/en/print.html; CDC. 2003. Revised US surveillance case definition for Severe Acute Respiratory Syndrome (SARS) and update on SARS cases—United States and Worldwide, December 2003. MMWR 52: 1202-1206.)
   CDC Laboratory Criteria
   RT-PCR results
2. To estimate the rate of indeterminate results from inhibitory specimens

Prior to commencement of the trial, approval of the protocol was obtained from the investigating site's Institutional Review Board. The trial site also demonstrated proficiency in performing the sample processing and assay procedures by testing a panel of mock specimens that were seeded with Ambion Armored RNA particles containing a cloned copy of the SARS-CoV nucleocapsid target sequence. The criteria for specimen enrollment are outlined in Table 8. All analysis using the BD ProbeTec ET System was conducted in a blinded fashion without prior knowledge of the operators to the results of RT-PCR or other predicate testing methods.

Specimens were processed using a QIAamp Viral RNA Mini Kit essentially according to the manufacturer's instructions except that an on-column DNase treatment was incorporated to remove contaminating DNA. For stool specimens, an additional pre-processing step was included to remove particulate matter prior to loading on the QIAGEN columns. Stool samples were diluted 1:10 with 0.89% saline and centrifuged for 20 min. at 4,000×g. The supernatant was then decanted and passed through a 0.22 μm filter to remove particulate debris.

In brief, 140 μL of the clinical specimen or stool filtrate were processed through a QIAamp column that was treated with DNase to digest contaminating non-specific DNA bound to the column matrix. After washing to remove the DNase, purified RNA was eluted in a volume of 80 μL water. Thirty microliters of eluate were added to a Priming Microwell containing dried primers, Reporter Probes and nucleotides, followed by 20 μL of Reverse Transcription Buffer containing RNase inhibitor, AMV-RT enzyme and 1000 copies of IAC RNA transcripts (SEQ ID NO.: 12). Final reaction conditions for reverse transcription were as follows: 1500 μM dCₛTP; 300 μM each of dATP, dGTP and dTTP; 5 mM magnesium acetate; 1500 nM bumper primer SARSrtB24 (SEQ ID NO.: 1); 1500 nM SDA Primer SarCRP (SEQ ID NO.: 5); 300 nM SDA Primer SarCFP (SEQ ID NO.: 4); 750 nM Signal Primer SarCAd-MPC (SEQ ID NO.: 8); 600 nM IAC Signal Primer SarC-IACAd (SEQ ID NO.: 7); 1200 nM target Reporter Probe MPC D/R (SEQ ID NO.: 15); 900 nM IAC Reporter Probe MPC2 F/D (SEQ ID NO.: 16); 1000 copies of IAC transcript (SEQ ID NO.: 12); 5% DMSO; 5% glycerol; 43.5 mM KᵢPO₄; 25 mM KOH; 120 mM bicine; 40U RNase inhibitor; 10U AMV-RT. Rehydrated microwells were then incubated at 48° C. for 20 min before addition of 100 μL of SDA Buffer and transfer to a 72° C. heat block. At the same time, Amplification Microwells containing dried SDA enzymes (Bst polymerase and BsoBI restriction enzyme) were pre-warmed at 54° C. After a 10 min. incubation, 100 μL of sample were transferred from the Priming Microwells to the Amplification Microwells, which were then sealed and incubated in a BD ProbeTec ET reader at 52.5° C. The final reaction conditions for SDA were as follows: 500 μM dCₛTP; 100 μM each of dATP, dGTP and dTTP; 5.7 mM magnesium acetate; 500 nM bumper primer SARSrtB24 (SEQ ID NO.: 1); 500 nM SDA Primer SarCRP (SEQ ID NO.: 5); 100 nM SDA Primer SarCFP (SEQ ID NO.: 4); 250 nM Signal Primer SarCAd-MPC (SEQ ID NO.: 8); 200 nM Signal Primer SarC-IACAd (SEQ ID NO.: 7); 400 nM Reporter Probe MPC D/R (SEQ ID NO.: 15); 300 nM Reporter Probe MPC2 F/D (SEQ ID NO.: 16); 12.5% DMSO; 1.7% glycerol; 24.5 mM KᵢPO₄; 82 mM KOH; 143 mM bicine; 12U Bst polymerase; 45U BsoBI restriction enzyme.

During the course of a 1 hour incubation, fluorescent readings were taken every minute in both optical channels of the BD ProbeTec ET instrument and results were reported in terms of the PAT scores for the SARS-CoV target and IAC. Reactions in which the fluorescent readings never achieved the predetermined threshold of fluorescence were assigned a PAT score of 0. Reactions that yielded ROX PAT scores >0, corresponding to the MPC D/R Reporter Probe (SEQ ID NO.: 15), were considered positive for SARS-CoV, while reactions that yielded FAM PAT scores >0, corresponding to the IAC Reporter Probe MPC2 F/D (SEQ ID NO.: 16), were considered positive for IAC. Those in which neither the FAM nor ROX signals achieved their respective thresholds (PAT scores=0) were considered indeterminate. External positive and negative controls were included in each assay run to verify performance. These controls were required to yield the positive and negative correct results respectively in order for the results from patient specimens to be reported by the instrument.

### Results and Conclusions

Results from the analysis of 30 stool specimens and 30 NP aspirates relative to those from a validated RT-PCR method are summarized in Tables 9 and 10. Pieris J S M et al., Lancet 361:1767-1772 (2003); Poon L L M, et al., Clin Chem 49: 953-955 (2003); Poon L L M et al. Clin Chem 50: 67-72 (2004); Poon L L M et al., J Clin Virol 238: 233-238 (2003). The stool specimens were collected between days 8 and 13 following the onset of SARS-like symptoms (median=11 days), while for NP aspirates the range was 3 to 7 days (median=5 days). The sensitivity, specificity and indeterminate rate for the BD ProbeTec SARS-CoV Assay with stool samples were 100%, 100% and 3.3% respectively, while for NP aspirates, sensitivity and specificity were both 100% and no indeterminate results were recorded. For both specimen types combined, sensitivity, specificity and indeterminate rate were therefore 100% (32/32), 100% (27/27) and 1.6% (1/60), respectively.

The data presented here demonstrate that the BD ProbeTec ET assay is both sensitive and specific for the detection of SARS-CoV in clinical specimens. Clinical sensitivity of the RT-SDA system was shown to be equivalent to that of a validated RT-PCR method, with the added benefit that the RT-SDA-based system incorporates an RNA-based IAC to monitor for inhibition of the assay and/or RNase contamination.

Detection of SARS-CoV in NP aspirates during the early stages of infection bodes well for the utility of the BD ProbeTec ET assay for patient management in terms of rapid therapeutic intervention and/or implementation of infection control measures, prior to dissemination of the virus within the community.

TABLE 8
Criteria for specimen enrollment

| Category | Inclusion Criteria | Exclusion Criteria |
|---|---|---|
| Patient | Retrospective specimens from patients | Retrospective |
| Population | with probable/suspect SARS and/or | specimens from |
| | laboratory confirmed SARS | patients that do |
| | | not have |
| | | probable/suspect |
| | | SARS |
| Specimen | Archived, frozen retrospective specimens | Prospective |
| Types | Respiratory specimens | specimens |
| | Stool/Rectal swab | Specimens other |
| | Serum | than respiratory, |
| | Residual purified eluate * | stool/rectal |
| | | swab, serum, |
| | | purified eluate * |
| Method | BD ProbeTec ET SARS CoV assay, | No RT-PCR |
| Types | required | result |
| | RT-PCR SARS-CoV assay, required | |
| | Serology (ELISA), if available | |
| | Culture results, if available | |
| | Immunofluorescent antigen assay, if | |
| | available | |

* Refers to processed specimen obtained from QIAGEN or other nucleic acid purification process

TABLE 9
Analysis of stool specimens with the BD ProbeTec
ET SARS-CoV assay relative to RT-PCR

| | RT-PCR * | | |
|---|---|---|---|
| BD ProbeTec | Positive | Negative | Total |
| Positive | 15 | 0 | 15 |
| Negative | 0 | 14 | 14 |
| Indeterminate | 0 | 1 | 1 |
| Total | 15 | 15 | 30 |

* The RT-PCR method does not incorporate an IAC to monitor for assay inhibition

TABLE 10
Analysis of NP aspirates with the BD ProbeTec
ET SARS-CoV assay relative to RT-PCR

| | RT-PCR * | | |
|---|---|---|---|
| BD ProbeTec | Positive | Negative | Total |
| Positive | 17 | 0 | 15 |
| Negative | 0 | 13 ** | 14 |
| Indeterminate | 0 | 0 | 1 |
| Total | 17 | 13 | 30 |

* The RT-PCR method does not incorporate an IAC to monitor for assay inhibition
** One specimen was from a patient who conformed to the CDC/WHO case definition for SARS

### SARS Assay System D Examples

### Example 1

### DNA Amplification Using SARS-CoV-Specific Primers

The ability of the disclosed combination of primers and probes to amplify SARS-CoV nucleic acid was demonstrated using a pUC19-based plasmid clone of the targeted region of the genome (corresponding to nucleotides 28996-29076 of SARS-CoV strain BJ03; GenBank Accession No. AY278490). The plasmid DNA was linearized with the restriction enzyme NarI and quantified using PicoGreen dsDNA Quantitation Reagent. Four replicate SDA reactions were run at each of three target levels, in addition to negative controls.

In brief, DNA target was added to bicine-based SDA Buffer and denatured by heating in a boiling water bath. One hundred and ten microliters of the denatured sample were then added to Priming Microwells containing 404 of the following mixture: 37.5 mM KᵢPO₄; 188 ng/g, BSA; 1900 μM dC₅TP; 375 μM each of dATP, dGTP and dTTP; 375 nM SDA Primer SarDFP (SEQ ID NO.: 18); 1875 nM SDA Primer SarDRP (SEQ ID NO.: 19); 938 nM Signal Primer SarDAd-TBD16 (SEQ ID NO.: 20); 1875 nM Reporter Probe TBD16 D/R (SEQ ID NO.: 13); 3.75 mM magnesium acetate. After 20 min at ambient temperature, the Priming Microwells were transferred to a heat block at 72° C., while corresponding Amplification Microwells containing dried Bst polymerase and BsoBI restriction enzyme were pre-warmed at 54° C. After a 10 min. incubation, 100 μL of the priming mixture were transferred from the Priming to the Amplification Microwells, which were then sealed and placed at 52.5° C. in a BD ProbeTec ET reader. Fluorescent signals were monitored over the course of 1 hour and analyzed using the PAT algorithm developed for this instrument. Final primer and probe concentrations in the SDA reactions were as follows: 500 nM SDA Primer SarDRP (SEQ ID NO.: 19); 100 nM SDA Primer SarDFP (SEQ ID NO.: 18); 250 nM Signal Primer SarDAd-TBD16 (SEQ ID NO.: 20); 500 nM Reporter Probe TBD16 D/R (SEQ ID NO.: 13).

### Results and Conclusions

Positive results were obtained from all reactions containing SARS-CoV target DNA (Table 11). In contrast, none of the negative controls yielded positive results, demonstrating that the disclosed combination of primers and Reporter Probe is capable of detecting the targeted SARS-CoV-specific nucleic acid target sequence with a high degree of analytical sensitivity.

TABLE 11
Amplification and detection of a
SARS-CoV-specific target sequence

| | PAT Score | | | | |
|---|---|---|---|---|---|
| Target Level | A | B | C | D | Mean |
| 1000 | 49.7 | 49.7 | 49.6 | 49.6 | 49.7 |
| 100 | 46.4 | 49.2 | 49.4 | 44.5 | 47.4 |
| 10 | 45.9 | 37.4 | 46.9 | 47.0 | 44.3 |
| 0 | 0 | 0 | 0 | 0 | 0 |

PAT Score >0 = Positive

1. A oligonucleotide set comprising a first amplification primer and a second amplification primer, the first amplification primer consisting essentially of SEQ ID NO.: 4 or 18 and the second amplification primer consisting essentially of SEQ ID NO.: 5 or 19.

2. The oligonucleotide set of claim 1 wherein the first amplification primer consists essentially of SEQ ID NO.: 4 and the second amplification primer consists essentially of SEQ ID NO.: 5 or the first amplification primer consists essentially of SEQ ID NO.: 18 and the second amplification primer consists essentially of SEQ ID NO.: 19.

3. (canceled)

4. A oligonucleotide set comprising a first amplification primer and a second amplification primer, the first amplification primer consisting essentially of the target binding sequence of SEQ ID NO.: 4 or 18 and the second amplification primer consisting essentially of the target binding sequence of SEQ ID NO.: 5 or 19.

5. The oligonucleotide set of claim 4 wherein the first amplification primer consists essentially of the target binding sequence of SEQ ID NO.: 4 and the second amplification primer consists essentially of the target binding sequence of SEQ ID NO.: 5 or the first amplification primer consists essentially of the target binding sequence of SEQ ID NO.: 18 and the second amplification primer consists essentially of the target binding sequence of SEQ ID NO.: 19.

6. (canceled)

7. The oligonucleotide set of claim 1, further comprising a signal primer and a reporter probe, the signal primer consisting essentially of the target binding sequence of SEQ ID NO.: 6, 8, 20 or 21 and the reporter probe consisting essentially of SEQ ID NO.: 13 or 15.

8. The oligonucleotide set of claim 7, wherein the signal primer consists essentially of the target binding sequence of SEQ ID NO.: 6 and the reporter probe consists essentially of SEQ ID NO.: 13, the signal primer consists essentially of the target binding sequence of SEQ ID NO.: 8 and the reporter probe consists essentially of SEQ ID NO.: 15, the signal primer consists essentially of the target binding sequence of SEQ ID NO.: 20 and the reporter probe consists essentially of SEQ ID NO.: 13, or the signal primer consists essentially of the target binding sequence of SEQ ID NO.: 21 and the reporter probe consists essentially of SEQ ID NO.: 15.

9. (canceled)

10. (canceled)

11. (canceled)

12. The oligonucleotide set of claim 7, further comprising one or more bumper primers consisting essentially of SEQ ID NO.: 1, 2, 3 or 17.

13. The oligonucleotide set of claim 8, further comprising a second signal primer and a second reporter probe, the second signal primer consisting essentially of SEQ ID NO.: 25 and the second reporter probe consisting essentially of the hybridization sequence of SEQ ID NO.: 14.

14. The oligonucleotide set of claim 13, further comprising one or more bumper primers consisting essentially of SEQ ID NO.: 1, 2, 3 or 17.

15. The oligonucleotide set of claim 8, wherein the signal primer consists essentially of the target binding sequence of SEQ ID NO.: 8 and the reporter probe consists essentially of SEQ ID NO.: 15 and further comprising a second signal primer and a second reporter probe, the second signal primer consisting essentially of SEQ ID NO.: 7 and the second reporter probe consisting essentially of the hybridization sequence of SEQ ID NO.: 16.

16. The oligonucleotide set of claim 15, further comprising one or more bumper primers consisting essentially of SEQ ID NO.: 1, 2, 3 or 17.

17. The oligonucleotide set of claim 4, wherein the target binding sequences of SEQ ID NOs.: 4, 5, 18 and 19 comprise a sequence required for an amplification reaction.

18. The oligonucleotide set of claim 17, wherein the sequence required for the amplification reaction comprises a restriction endonuclease recognition site that is nickable by a restriction endonuclease or a promoter recognized by an RNA polymerase.

19. (canceled)

20. The oligonucleotide set of claim 7, wherein the hybridization sequences of SEQ ID NOs.: 6, 8, 13, 15, 20 and 21 further comprise an indirectly detectable marker.

21. (canceled)

22. The oligonucleotide set of claim 4, further comprising a signal primer and a reporter probe, the signal primer consists essentially of the target binding sequence of SEQ ID NO.: 6, 8, 20 or 21 and the reporter probe consists essentially of SEQ ID NO.: 13 or 15.

23. The oligonucleotide set of claim 22, wherein the signal primer consists essentially of the target binding sequence of SEQ ID NO.: 6 and the reporter probe consists essentially of SEQ ID NO.: 13, the signal primer consists essentially of the target binding sequence of SEQ ID NO.: 8 and the reporter probe consists essentially of SEQ ID NO.: 15, the signal primer consists essentially of the target binding sequence of SEQ ID NO.: 20 and the reporter probe consists essentially of SEQ ID NO.: 13, or the signal primer consists essentially of the target binding sequence of SEQ ID NO.: 21 and the reporter probe consists essentially of SEQ ID NO.: 15.

24. (canceled)

25. (canceled)

26. (canceled)

27. The oligonucleotide set of claim 22, further comprising one or more bumper primers consisting essentially of SEQ ID NO.: 1, 2, 3 or 17.

28. The oligonucleotide set of claim 23, further comprising a second signal primer and a second reporter probe, the second signal primer consisting essentially of SEQ ID NO.: 25 and the second reporter probe consisting essentially of the hybridization sequence of SEQ ID NO.: 14.

29. The oligonucleotide set of claim 28, further comprising one or more bumper primers consisting essentially of SEQ ID NO.: 1, 2, 3 or 17.

30. The oligonucleotide set of claim 23, wherein the signal primer consists essentially of the target binding sequence of SEQ ID NO.: 8 and the reporter probe consists essentially of SEQ ID NO.: 15, and further comprising a second signal primer and a second reporter probe, the second signal primer consisting essentially of SEQ ID NO.: 7 and the second reporter probe consisting essentially of the hybridization sequence of SEQ ID NO.: 16.

31. The oligonucleotide set of claim 30, further comprising one or more bumper primers consisting essentially of SEQ ID NO.: 1, 2, 3 or 17.

32. The oligonucleotide set of claim 4, wherein the target binding sequence of SEQ ID NOs.: 4, 5, 18 and 19 comprises a sequence required for an amplification reaction.

33. The oligonucleotide set of claim 32, wherein the sequence required for the amplification reaction comprises a restriction endonuclease recognition site that is nickable by a restriction endonuclease or a promoter recognized by an RNA polymerase.

34. (canceled)

35. The oligonucleotide set of claim 32, wherein the hybridization sequences of SEQ ID NOs.: 6, 8, 13, 15, 20 and 21 further comprise an indirectly detectable marker.

36. (canceled)

37. An oligonucleotide comprising a SARS Coronavirus (SARS-CoV) target sequence consisting essentially of SEQ ID NO.: 9, 10, 22 or 23.

38. A method for detecting the presence or absence SARS-CoV in a sample, the method comprising:
(a) treating the sample with a plurality of nucleic acid primers in a nucleic acid amplification reaction wherein a first primer consists essentially of the target binding sequence of SEQ ID NO.: 4 or 18 and a second primer consists essentially of the target binding sequence of SEQ ID NO.: 5 or 19; and
(b) detecting any amplified nucleic acid product, wherein detection of the amplified product indicates presence of SARS CoV.

39-45. (canceled)

46. A method for amplifying a target nucleic acid sequence of SARS-CoV comprising:
(a) hybridizing to the nucleic acid
(i) a first amplification primer consisting essentially of the target binding sequence of SEQ ID NO.: 4 or 18; and
(ii) a second amplification primer consisting essentially of the target binding sequence of SEQ ID NO.: 5 or 19; and
(b) extending the hybridized first and second amplification primers on the target nucleic acid sequence whereby the target nucleic acid sequence is amplified.

47-57. (canceled)

58. A method of quantifying the amount of SARS-CoV nucleic acid in a target sample comprising the steps of:
a) combining the target sample with a known concentration of SARS-CoV internal control nucleic acid;
b) amplifying the target nucleic acid and internal control nucleic acid in an amplification reaction;
c) detecting the amplified nucleic acid; and
d) analyzing the relative amounts of amplified SARS-CoV target nucleic acid and internal control nucleic acid.

59. The method of claim 58, wherein the amplification reaction utilizes one or more signal primers consisting essentially of the hybridization sequence of SEQ ID NO.: 6, 7, 8, 20, 21 or 25 and one or more reporter probes consisting essentially of the hybridization sequence of SEQ ID NO.: 13, 14, 15 or 16.

60. The method of claim 59, wherein the hybridization sequences of SEQ ID NOs.: 6, 7, 8, 13, 14, 15, 16, 20, 21, and 25 comprise an indirectly detectable marker.

61-63. (canceled)
